(19) United States
(12) Patent Application Publication
(10) Pub. No.: US 2012/0003182 A1
(43) Pub. Date: Jan. 5, 2012

(54) GENETIC SEVERITY MARKERS IN MULTIPLE SCLEROSIS

(75) Inventors: Hadi Abderrahim, Divonne Les Bains (FR); Jérôme Wojcik, Divonne Les Bains (FR); Federica Esposito, Sotto II Monte Giovanni XXIII (BG) (IT); Virginie Debailleul, Peron (FR)

(73) Assignee: MERCK SERONO SA, Coinsins, Vaud (CH)

(21) Appl. No.: 13/147,653

(22) PCT Filed: Mar. 25, 2010
(86) PCT No.: PCT/EP10/53871

§ 371 Date: Aug. 3, 2011

Related U.S. Application Data
(60) Provisional application No. 61/165,141, filed on Mar. 31, 2009.

(30) Foreign Application Priority Data
Mar. 27, 2009 (EP) 09156487.2

(51) Int. Cl. A61K 38/21; A61P 25/00; C12Q 1/68

(52) U.S. Cl. 424/85.6; 435/6.11

(57) ABSTRACT

The present invention relates to the use of SNPs in predicting susceptibility and/or severity of Multiple Sclerosis in an individual. The SNPs are located in the introns of the glycosylation enzymes MGAT5 and XYLT1, 3′ of HIF1AN, within introns of MEGF11. FGF14, PDE9A and CDH13 and within desert regions of 4q34 and 17p13.

### FIELD OF THE INVENTION

The present invention relates to the use of SNPs to identify an association with the severity of Multiple Sclerosis (MS) in a subject.

### BACKGROUND OF THE INVENTION

Multiple Sclerosis (MS) is a chronic inflammatory and demyelinating disease of the Central Nervous System (CNS), often starting in early adulthood. MS is considered a complex disease, since multiple genetic and non-genetic factors are likely to combine to influence the risk to disease. The evidence for a role of genetic factors is compelling and is supported by twin, half-sibling and adoptee studies. Whereas MS usually starts with a relapsing-remitting course (RR), most patients later enter a secondarily progressive phase (SP) while others, often with a later onset, may enter directly into primary progression (PP).

Genome scans have excluded the presence of a major susceptibility locus in MS apart from the HLA class II region, and failed to reveal more than a few putative susceptibility loci¹⁻³. Within the HLA gene complex, associations with several alleles of HLA-DRB1 have been indicated⁴, whereas some evidence also suggests an independent factor for risk of MS in the HLA class I region⁵⁻⁷. Very recently, evidence supporting an importance of the IL7Ra gene in MS is mounting⁸⁻¹⁰. However, it is clear that other genetic risk factors remain to be identified.

Susceptibility to MS is unequivocally a complex genetic trait. Clinical course and outcome of MS differ widely and it seems likely that while some genes may be involved in the induction of the disease, others may have a role in influencing the disease severity^{11,12}. Severity in MS is assessed as development of disability as a function of duration of disease but may be complicated by the fact that the rate of progression differs from time to time and that patients may also show periods of improvement. The most widely used method of clinical assessment of MS severity is based on the Expanded Disability Status Scale (EDSS¹³). Traditionally, the Progression Index (PI=EDSS score/duration in years) has been widely used but is hampered by the reasons mentioned above. More recently the MS Severity Score (MSSS) has been proposed as a novel approach, relating scores on the EDSS to the distribution of disability in patients with comparable disease durations, partly compensating for the weaknesses of the PI¹⁴.

Several candidate genes have been tested for a possible association with MS severity (see¹⁵ for review). Most of them displayed no evidence of genetic association with MS prognosis: apolipoprotein ε (APOE, see¹⁶ for review), spinocerebellar ataxia 2 (SCA2¹⁷), brain-derived neurotrophic factor (BDNF¹⁸), toll-like receptor 4 (TLR4¹⁹), osteopontin²⁰, cytotoxic T lymphocyte associated 4 (CD152 or CTLA4) and CD28²¹, and chemokine CC receptor 5 (CCR5) and HLA-DRB1*1501²². Only a handful of loci have been reported to be associated with the clinical outcome of MS: the interleukin-1 locus on chromosome 2q12-14 contains 3 genes (IL-1α, IL-1β and IL-1 receptor antagonist IL-1RN) in which 6 sites, 5 single nucleotide polymorphisms (SNPs) and one variable number tandem repeat (VNTR), were reported to be associated with severity measured by EDSS graded in three severity categories²³ ; in the interleukin-10 promoter, two microsatellite markers were reported as differentially represented between mild (PI<0.5) and severe (PI>0.5) disease progression categories²⁴ ; two SNPs have been found associated with MS categories (relapsing remitting—RR vs. primary progressive—PP) but not with prognosis measured by MSSS in the ADAMTS14 gene²⁵ ; and disease incidence and severity have been shown to be increased in CD59a-deficient in MOG-EAE murine model²⁶. However, these studies were based on limited numbers of individuals and none was replicated. In addition, all these studies used categorical approaches to detect association with severity: the patients are categorized in mild/moderate/severe or mild/severe MS forms by choosing cutoff thresholds on the lesion volumes, the EDSS, the PI or the MSSS scales, and frequencies of alleles and genotypes are compared between categories.

In view of the significance of MS there is a need to identify markers, in particular genetic markers, useful in MS patients. In particular there exists a need to identify genetic markers useful predicting susceptibility and in particular severity of the disease MS.

### SUMMARY OF THE INVENTION

The present invention in one aspect is directed to a method for genotyping comprising the steps of a. using a nucleic acid isolated from a sample of an individual; and b. determining the type of nucleotide in SNP rs3814022, rs4953911, rs2059283, rs12927173, rs2495725, rs1343522, rs4573623 rs333548, rs10508075, rs2839580, rs2495725, rs3814022, rs1078922, and/or rs4315313 in one or both of the alleles of the diallelic marker, and/or in a SNPs in Linkage Disequilibrium (LD) with one or more of these SNPs.

In one aspect the invention relates to one or more SNPs selected from the group consisting of SNPs rs3814022, rs4953911, rs2059283, rs12927173, rs2495725, rs1343522, rs4573623, rs333548, rs10508075, rs2839580, rs2495725, rs3814022, rs1078922, rs4315313, SNPs in Linkage Disequilibrium (LD) with one or more of these SNPs for use in predicting in an individual the severity of the disease Multiple Sclerosis.

In yet another aspect the invention relates to a method for treating Multiple Sclerosis in an individual in need thereof, the method comprising the steps of a. applying a method as described above to a sample of an individual in vitro; b. treating said individual identified to exhibit one or more of the markers described above and which individual has been identified to exhibit a certain level of severity of the disease Multiple Sclerosis.

### DETAILED DESCRIPTION OF THE INVENTION

In the following the invention will be described in more detail wherein the examples are intended to illustrate the invention without being construed to be limiting the scope of the invention.

### BRIEF DESCRIPTION OF THE TABLES AND FIGURES

FIG. 1. MSSS distribution.

Histogram of the MS Severity Score distribution over the 1,040 MS patients.

FIG. 2. FDR (False Discovery Rate) estimation of severity associations.

The FDR was estimated with 10,000 rounds of MSSS shuffling and plotted against the number of selected positives R, for R≦100 (thick line). This curve gives the estimated proportion of false-positives for a given number of positives (e.g. 90% of the 40 most likely associated SNPs (R≦40) are estimated to be false-positives) or the number of positives for a given false-discovery rate (e.g. only one SNP is selected at 40% FDR threshold). Dashed lines represent the boundaries of the 95% estimation confidence interval.

FIG. 3. Examples of SNP associated with disease severity.

The scatter plots on the left represent the MSSS distributions in the whole population (black: (1) far left column) and for the individuals having the major homozygote (red: (2) 2^{nd} column from left), the heterozygote (blue: 3^{rd} column from left) and the minor homozygote (green: far right column) for the considered SNP. Horizontal lines (resp. boxes) indicate MSSS averages (resp. standard deviations) within categories. Cumulative distribution functions are represented on the right.

FIG. 3.1: .SNP 1 desert chr4, rs6552511

FIG. 3.2: SNP 2 desert chr17, rs7221818

FIG. 3.3: SNPs 3 and 4. XYLT1, rs12927173 and rs2059283

FIG. 3.4: SNPs 5, 7 and 11. HIF1AN, rs1343522, rs4573623 and rs2495725

FIG. 3.5: SNPs 6 and 12. MGAT5, rs4953911 and rs3814022

FIG. 3.6: SNP 8. MEGF11, rs333548

FIG. 3.7: SNP 9. FGF14, rs10508075

FIG. 3.8: SNP 10. PDE9A, rs2839580

FIG. 3.9: SNP 13. MTPN, rs1078922

FIG. 3.10: SNP 14. CDH13, rs4315313

FIG. 4. Replication of SNPs in XYLT1 and MGAT5.

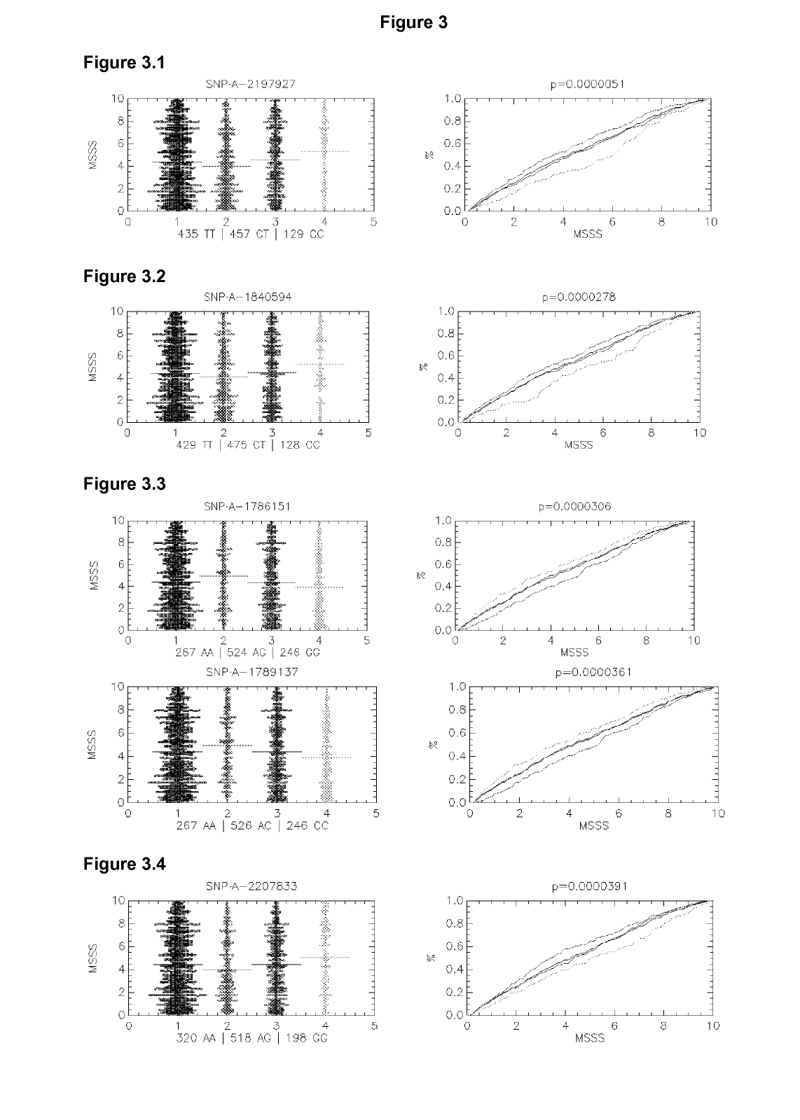

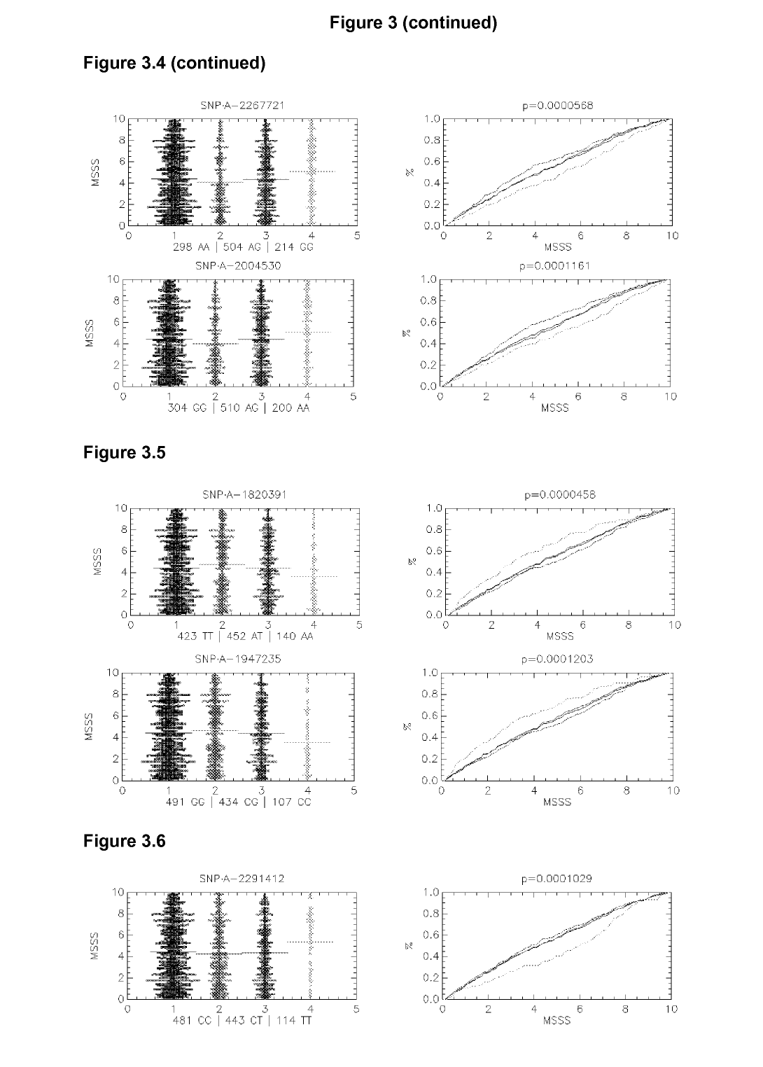

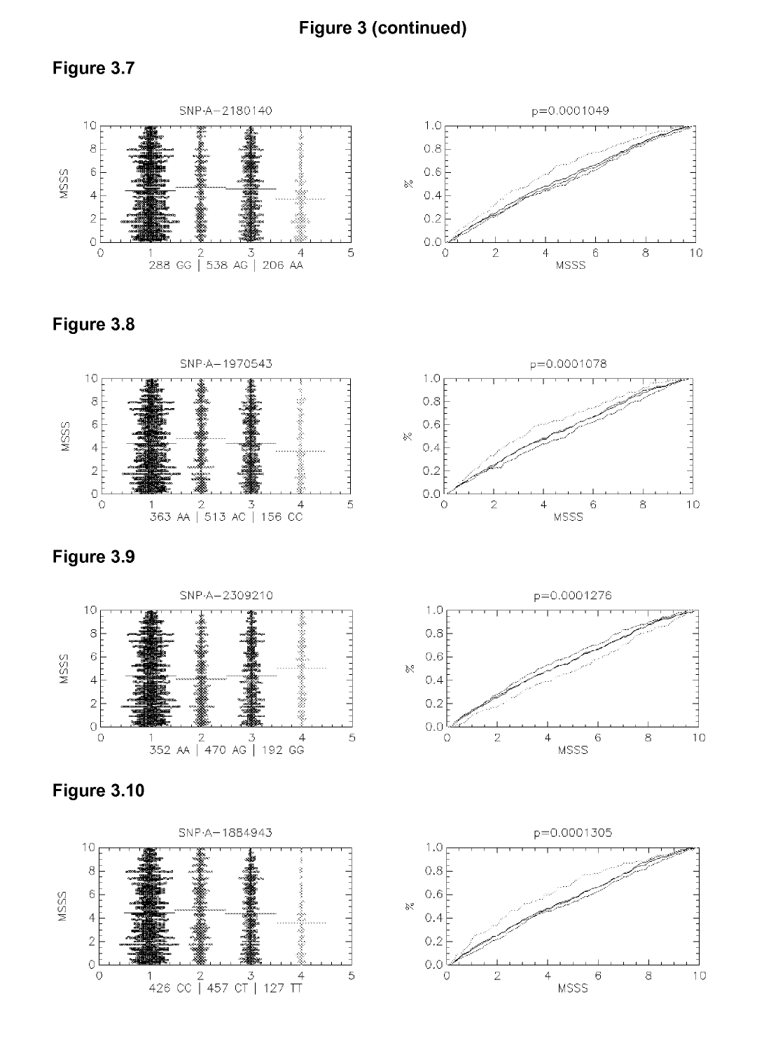

Association scatter plots (same legend as in FIG. 3) of 3 SNPs on the replication dataset of 873 independent samples. The first two top SNPs are located in the MGAT5 gene and the third one is in the XYLT1 gene.

The SNPs and context sequences are depicted in the following

| | | | | | | SNP | | |
|---|---|---|---|---|---|---|---|---|
| SEQ | | | | | Affymetrix | sequence | | |
| ID | | Affymetrix | | Position | probe | (Affymetrix | Severity | 2nd |
| No. | SNP | ID | chromosome | Build 36 | orientation | probe) | allele | allele |
| 1 | rs6552511 | SNP_A-2197927 | 4 | 182,688,603 | reverse | CATTGCAACTCATCTAY | C | T |
| | | | | | | ACCTGTAACTCTTGTT | | |
| 2 | rs7221818 | SNP_A-1840594 | 17 | 5,817,571 | reverse | TAGCCGTTGTTGTCCAY | C | T |
| | | | | | | CTCCTCCAATAGAATG | | |
| 3 | rs12927173 | SNP_A-1786151 | 16 | 17,378,819 | reverse | GGCTGGCTGTCCCGCCR | A | G |
| | | | | | | AACAAAGAGCCTGGAT | | |
| 4 | rs2059283 | SNP_A-1789137 | 16 | 17,376,995 | forward | TTGACCAGCCTTATCAM | A | C |
| | | | | | | ATCTGACTGTATTTCC | | |
| 5 | rs1343522 | SNP_A-2207833 | 10 | 102,358,149 | reverse | CCCAAAGATGCCGGACR | G | A |
| | | | | | | GATACCCCAAGAGGTG | | |
| 6 | rs4953911 | SNP_A-1820391 | 2 | 134,785,264 | reverse | GTTTATAAAAACTCTCW | T | A |
| | | | | | | GAAACCTCAAAGAACA | | |
| 7 | rs4573623 | SNP_A-2267721 | 10 | 102,361,371 | reverse | GAATCAGGTTCTGATCR | G | A |
| | | | | | | AGATCCACAAATTTTA | | |
| 8 | rs333548 | SNP_A-2291412 | 15 | 64,032,551 | forward | GCAATTACCGGTAAGCY | T | C |
| | | | | | | ATGAGAGTAGTGGGGG | | |
| 9 | rs10508075 | SNP_A-2180140 | 13 | 101,237,184 | forward | TGTTGCTGACAATTAAR | G | A |
| | | | | | | CCACATAGCATTTATA | | |
| 10 | rs2839580 | SNP_A-1970543 | 21 | 43,030,160 | reverse | TTGCATCTTTGGGTTAM | A | C |
| | | | | | | GGCTCTGCTGCCCTTG | | |
| 11 | rs2495725 | SNP_A-2004530 | 10 | 102,353,994 | reverse | AGTCCCTAAGTGCCACR | A | G |
| | | | | | | AATGAAAAGAAGACTC | | |
| 12 | rs3814022 | SNP_A-1947235 | 2 | 134,764,389 | forward | TTTAATTCCCCACAAAS | G | C |
| | | | | | | AGCTGAGTGGCTCTTG | | |
| 13 | rs1078922 | SNP_A-2309210 | 7 | 135,334,923 | reverse | GGAAAACAAATTTTCCR | G | A |
| | | | | | | CTTCTAAGGCTGTTAA | | |
| 14 | rs4315313 | SNP_A-1884943 | 16 | 81,644,218 | forward | TGAATGAGATAATTCAY | C | T |
| | | | | | | GTGAGGCTCTTAGAAA | | |

IUPAC SNP Codes:

| IUPAC Code | SNP |
|---|---|
| R | G or A |
| Y | T or C |
| M | A or C |
| K | G or T |
| S | G or C |
| W | A or T |

The present invention in one aspect is directed to a method for genotyping comprising the steps of a. using a nucleic acid isolated from a sample of an individual; and b. determining the type of nucleotide in SNP rs3814022, rs4953911, rs2059283, rs12927173, rs2495725, rs1343522, rs4573623 rs333548, rs10508075, rs2839580, rs2495725, rs3814022, rs1078922, and/or rs4315313 in one or both of the alleles of the diallelic marker, and/or in a SNPs in Linkage Disequilibrium (LD) with one or more of these SNPs.

SNPs of particular interest are preferably selected from rs3814022, rs4953911, rs2059283, rs12927173, rs2495725, rs1343522 and/or rs4573623.

In one embodiment, SNPs according to the invention and useful in the methods and uses of the invention are also those SNPs in Linkage Disequilibrium (LD) with one or more of the identified SNPs, as expressed by a LD correlation coefficient r² greater than 0.8 in at least one population of at least 100 individuals, preferably a LD correlation coefficient r² greater than 0.95.

“Association” of a marker e.g. a SNP with the severity in a Multiple Sclerosis patient according to the invention means the statistically significant difference of marker frequencies between two populations of patients having different severity levels of Multiple Sclerosis.

“Severity” of Multiple Sclerosis (MS) may be expressed according to the invention with any means known in the field of MS like e.g. Expanded Disease Status Scale (EDSS) or with other commonly used techniques or measurements or definitions in the field. The term “residual disease activity” frequently used in this context and in the filed is to be understood as indicating a certain level of MS disease activity, e.g. showing clinical symptoms, as defined by any of the measurements or definitions usually applied in the field of MS. One indicator or measurement of “residual disease activity” can be the experience of relapse(s) or disease progression as e.g. measured by Expanded Disease Status Scale (EDSS) or Magnetic Resonance Imaging (MRI). As time frame one example is the assessment during two years of treatment. It is appreciated that other time frames may be defined and used, e.g. one year, three years, or others as usually applied in clinical study protocols and well known to the skilled person. The time frame of reference may be chosen so as to allow for a measurement and appropriate read-out. Equally applicable, other accepted disease status measurements may be applied as e.g. The Cambridge Multiple Sclerosis Basic Score (CAMBS) and others used by the skilled person. There exist various definitions of an MS attack in the field and as understood by the skilled person in the field of MS that may be applied according to the invention. Accordingly, various possibilities exist for the skilled person that can be applied when working the invention. Examples of the assessment or diagnosis of MS are published in Kurzke J. F., Neuroepidemiology, 1991, 10: 1-8 ; Kurzke J. F., Neurology, 1983, 33: 1444-1452 ; McDonald W. I et al., Ann. Neurol., 2001, 50: 121-127 ; Polman C. H. et al., Ann. Neurol. 2005, 58 : 840-846. Accordingly, a severity marker or SNP may represent a marker indicating high disease or low disease severity in a patient as compared to the MS population.

An individual treated according to the invention will “respond” to treatment. “Response” or “responders” to interferon treatment in an individual diagnosed as having MS, suffering from MS or a MS patient in the sense of the present invention is understood to be residual disease activity according to the criteria set out below upon interferon treatment, in particular with interferon-beta 1a or 1b, and in particular Rebif®, Avonex®, Cinnovex® Betaseron® and Extavia®, of a MS patient. The response may be defined and/or measured as increase in time to the progression of the disease as measured by e.g. Expanded Disease Status Scale (EDSS) or with other commonly used techniques or measurements or definitions in the field. In particular it is to be understood as non-progression or non-worsening of MS or a stable clinical profile/activity or as the improvement of MS in e.g. clinical signs or measured with other means as e.g. MRI or CSF (cerebrospinal fluid) analysis. In particular it may be understood as less frequent relapses/attacks/exacerbation or milder relapses/attacks/exacerbation.

As used in the specification and the claims, “a” or “an” means one or more unless explicitly stated otherwise.

An “allele” is a particular form of a gene, genetic marker or other genetic locus, that is distinguishable from other forms of the gene, genetic marker or other genetic locus; e.g. without limitation by its particular nucleotide sequence. The term allele also includes for example without limitation one form of a single nucleotide polymorphism (SNP). An individual can be homozygous for a certain allele in diploid cells; i.e. the allele on both paired chromosomes is identical; or heterozygous for said allele; i.e. the alleles on both paired chromosomes are not identical.

A “genetic marker” is an identifiable polymorphic genetic locus. An example without limitation of a genetic marker is a single nucleotide polymorphism (SNP). A “marker” may be a genetic marker or any other marker, e.g. the expression level of a particular gene on nucleotide level as mRNA, useful in the context of the invention to be indicative of a response to interferon treatment.

A “genotype” as used herein refers to the combination of both alleles of a genetic marker, e.g. without limitation of an SNP, on a single genetic locus on paired (homologous) chromosomes in an individual. “Genotype” as used herein also refers to the combination of alleles of more than one genetic loci, e.g. without limitation of SNPs, on a pair or more than one pair of homologous chromosomes in an individual.

“Genotyping” is a process for determining a genotype of an individual.

“Locus” or “genetic locus” refers to a specific location on a chromosome or other genetic material.

“Oligonucleotide” refers to a nucleic acid or a nucleic acid derivative; including without limitation a locked nucleic acid (LNA), peptide nucleic acid (PNA) or bridged nucleic acid (BNA); that is usually between 5 and 100 contiguous bases in length, and most frequently between 5-40, 5-35, 5-30, 5-25, 5-20, 5-15, 5-10, 10-50, 10-40, 10-30, 10-25, 10-20, 15-50, 15-40, 15-30, 15-25, 15-20, 20-50, 20-40, 20-30 or 20-25 contiguous bases in length. The sequence of an oligonucleotide can be designed to specifically hybridize to any of the allelic forms of a genetic marker; such oligonucleotides are referred to as allele-specific probes. If the genetic marker is an SNP, the complementary allele for that SNP can occur at any position within an allele-specific probe. Other oligonucleotides useful in practicing the invention specifically hybridize to a target region adjacent to an SNP with their 3′ terminus located one to less than or equal to about 10 nucleotides from the genetic marker locus, preferably 5 about 5 nucleotides. Such oligonucleotides hybridizing adjacent to an SNP are useful in polymerase-mediated primer extension methods and are referred to herein as “primer-extension oligonucleotides.” In a preferred embodiment, the 3′-terminus of a primer-extension oligonucleotide is a deoxynucleotide complementary to the nucleotide located immediately adjacent an SNP.

“Polymorphism” refers of two or more alternate forms (alleles) in a population of a genetic locus that differ in nucleotide sequence or have variable numbers of repeated nucleotide units. Polymorphisms occur in coding regions (exons), non-coding regions of genes or outside of genes. The different alleles of a polymorphism typically occur in a population at different frequencies, with the allele occurring most frequently in a selected population sometimes referenced as the “major” allele. Diploid organisms may be homozygous or heterozygous for the different alleles that exist. A diallelic polymorphism has two alleles. In said method preferably the identity of the nucleotides at said diallelic markers is determined for both copies of said diallelic markers present in said individual's genome. Any method known to the skilled person may be applied, preferably said determining is performed by a microsequencing assay. Furthermore, it is possible to amplify a portion of a sequence comprising the diallelic marker prior to said determining step, e.g. by PCR. However, any applicable method can be used.

It is preferred according to the invention that the method further comprises the step of correlating the result of the genotyping steps with associating the results with the severity of the disease Multiple Sclerosis.

It has now been found by the inventors in a preferred method according to the invention that the presence of a severity allele is characterized in rs3814022 by G, in rs4953911 by T, in rs2059283 by A, in rs12927173 by A, in rs2495725 by A, in rs1343522 by G, in rs4573623 by G, a T in rs333548, a G in rs10508075, a A in rs2839580, a A in rs2495725, a G in rs3814022, a Gin rs1078922, and/or a C in rs4315313 and that it is indicative of the severity of the disease Multiple Sclerosis. In the particular SNP according to the invention the respective base, A, T, C, G is present in one allele or preferably in both alleles and accordingly is indicative of the severity of MS. In particular, a SNP of the invention can indicate that an individual is probably more severly affected by MS or can represent a marker being indicative of being less severely affected by MS as compared to the average MS population.

The inventors thus advantageously provide for a means to make a distinction between different patients and different patient groups of the overall MS population and in particular classify them according to severity of the disease. In this means state of the art molecular biology methods and apparatus are applied like PCR and PCR cyclers, and alghorithms of statistics generally known to the person skilled in the art. The patients may thus be grouped as to their expected MS severity according to the MSSS in e.g. very severe, medium severe, not very severe and slightly severe. The invention hence provides for a tool that has implications for handling such patients better according to their stage and severity of disease. In particular it now will be possible to adapt the treatment dosage and treatment scheme better on an individual patient level.

In one preferred aspect the invention relates to one or more SNPs selected from the group consisting of rs3814022, rs4953911, rs2059283, rs12927173, rs2495725, rs1343522, rs4573623, rs333548, rs10508075, rs2839580, rs2495725, rs3814022, rs1078922, rs4315313, SNPs in Linkage Disequilibrium (LD) with one or more of these SNPs for use in predicting the severity of the disease Multiple Sclerosis in an individual.

In another aspect the invention is directed to a method for predicting the severity of the disease Multiple Sclerosis in an individual comprising a. using the nucleic acid from a sample of said individual; b. identifying the presence of a useful genetic marker in said individual by known methods; c. based on the results of step b) making a prediction of the severity of the disease Multiple Sclerosis for said individual.

In said method the genetic marker relates to one or more SNPs selected from the group consisting of rs3814022, rs4953911, rs2059283, rs12927173, rs2495725, rs1343522, rs4573623, rs333548, rs10508075, rs2839580, rs2495725, rs3814022, rs1078922, rs4315313, SNPs in Linkage Disequilibrium (LD) with one or more of these SNPs. SNPs of particular interest are preferably selected from rs3814022, rs4953911, rs2059283, rs12927173, rs2495725, rs1343522 and/or rs4573623.

In yet another aspect the invention relates to a method for treating Multiple Sclerosis in an individual in need thereof, the method comprising the steps of a. applying a method as described above to a sample of an individual; b. treating said individual by applying an interferon which individual has been identified by either of the above described methods as exhibiting one or more of the described markers and being at risk of having or developing a severe form of the disease Multiple Sclerosis. Alternatively, the invention relates to the use of an interferon for treating or interferon for the use in treating a Multiple Sclerosis patient which patient is characterized by carrying or has been identified to exhibit at least one severity allele of a SNP according to the invention. In a further alternative the invention relates to a SNP according to the invention for use in the diagnosis of MS severity in a patient and adopting the treatment of said patient according to his disease severity.

SNPs of particular interest in said method or use are preferably selected from rs3814022, rs4953911, rs2059283, rs12927173, rs2495725, rs1343522 and/or rs4573623.

The invention thus may be used in particular advantageously to stratify and adjust the interferon dose and/or the time point of treatment. A possible measure may be a high dose treatment or a treatment before clinical signs of MS are visible in a patient identified as highly severe affected by MS. MRI may be applied to analyze a patient's disease status and a grouping/classification of the patient according to these results may be performed in a manner pointed out above. It will be particularly advantageous for an MS patient identified according to the invention to have a high risk to be a MS patient who will be severely affected by the disease, to be treated at an early time point in order to manage the disease early on. Thus, appropriate measures like an adequate interferon treatment and dosage can be chosen. In addition the awareness of the patient will support compliance with the treatment. An increased compliance has in turn positive effects on the treatment results as such and its efficacy.

Preferably the interferon-beta (IFN) is interferon-beta la or 1b. Examples of interferon-beta are Rebif®, Avonex®, Cinnovex®, Betaseron® or Extavia®.

The dosage of IFN administered in the above method or use, as single or multiple doses, to an individual will vary in addition to the results of the patient grouping depending upon a variety of factors, including pharmacokinetic properties, the route of administration, patient conditions and characteristics (sex, age, body weight, health, size), extent of symptoms, concurrent treatments, frequency of treatment and the effect desired.

Standard dosages of human IFN-beta range from 80 000 IU/kg and 200 000 IU/kg per day or 6 MIU (million international units) and 12 MIU per person per day or 22 to 44 μg (microgram) per person. In accordance with the present invention, IFN may preferably be administered at a dosage of about 1 to 50 μg, more preferably of about 10 to 30 μg or about 10 to 20 μg per person per day.

The administration of active ingredients in accordance with the present invention may be by intravenous, intramuscular or subcutaneous route. A preferred route of administration for IFN is the subcutaneous route.

IFN may also be administered daily or every other day, of less frequent. Preferably, IFN is administered one, twice or three times per week

A preferred route of administration is subcutaneous administration, administered e.g. three times a week. A further preferred route of administration is the intramuscular administration, which may e.g. be applied once a week.

Preferably 22 to 44 μg or 6 MIU to 12 MIU of IFN-beta is administered three times a week by subcutaneous injection. IFN-beta may be administered subcutaneously, at a dosage of 25 to 30 μg or 8 MIU to 9.6 MIU, every other day. 30 μg or 6 MIU IFN-beta may further be administered intramuscularly once a week.

### EXAMPLES

The following examples are not meant to be construed limiting for the invention. The following examples represent preferred embodiments of the invention, which shall serve to illustrate the invention.

The examples show in a preferred embodiment of the invention the results of an approach to identify severity markers that is (i) genome-wide (i.e. hypothesis-free) and (ii) non categorical (i.e. continuous). First, three cohorts of MS patients (n=1,040) were recruited from hospitals in France, Sweden and Italy, and genotyped for about 500,000 SNPs genome-wide with the Affymetrix Genechip® 500K technology. MS severity was continuously scored by MSSS, and correlation with genotypes of the most frequent polymorphisms (˜105,000 SNPs) was evaluated by a non-parametric test between the MSSS distributions in patients homozygous for the alleles of each marker. The multiple-testing problem was controlled by False-Discovery Rate (FDR) estimation. The approach resulted in the identification of 14 severity markers, located in 8 different genes and 2 desert regions. Second, some markers have been genotyped on an independent replication cohort of 873 MS patients. Two glycosylation enzyme genes have been identified, thus supporting the importance of glycan regulation in MS.

### Materials & Methods

Collections

A total number of 1,040 unrelated patients from France, Italy and Sweden were included in the ‘screening’ dataset and 873 unrelated and independent patients from France and Sweden in the ‘replication’ dataset (Table 1). All the subjects were Caucasians and had a diagnosis of Multiple Sclerosis according to McDonald's criteria²⁷ and their disease courses were classified as either relapsing-remitting, secondary progressive or primary progressive²⁸. Disability was scored using the Kurtzke EDSS. The mean age was 43.8 years, the mean EDSS score was 3.6 and the sex ratio was 2.1 females/males. Informed consent for the genetic analysis was obtained from all individuals and local ethical committees approved the study protocol.

The detailed demographic and clinical characteristics of MS patients are shown in Table 2 for the screening and replication datasets. The disease duration has been defined as the number of years between the year of onset of first symptom and the year of last examination with EDSS assessment, in most cases at entry in the study. The age at onset was defined as the first episode of neurological dysfunction suggestive of demyelinating disease.

Grading of Disability

The Kurtzke EDSS is the most widely used measure of disability in MS studies, but it does not take into account the disease duration, a parameter that is critical in describing the rate of progression. For this reason we used the MSSS¹⁴, which provides a measure for disease severity in an individual patient on a cross-sectional basis. This scale relates scores on the EDSS to the distribution of disability in a large dataset of patients with comparable disease durations. The MSSS is computed using the MSSStest software program v2.0 described in¹⁴.

Genotyping & Quality Control

DNA samples of the screening dataset have been studied independently using the Affymetrix GeneChip® human mapping 500K technology. Genotypes of the 497,641 SNPs selected by Affymetrix were called for each DNA sample with the B-RLMM software program, ensuring a minimal call rate of 97%. Only SNPs from autosomal chromosomes were kept for analysis. In order to avoid biases due to very low genotype frequencies, markers with low Minor Allele Frequency (MAF<30%) or high rate of missing data (proportion of untyped DNA>5%) were filtered out. We chose to focus on very frequent markers (MAF>30%), which ensure a minimum minor homozygote frequency greater than 9% under Hardy-Weinberg equilibrium (and then a minor homozygote population size greater than 100 on average). DNA samples of the replication dataset have been genotyped independently for selected SNPs using Applied BioSystems TaqMan® genotyping assay.

Severity Scan

For every SNP, a Wilcoxon rank-sum test²⁹ was performed on the two sets of MS Severity Scores corresponding to patients homozygous for the alleles of each marker. This non-parametric test assigns a probability value (p-value) to every SNP. For the screening dataset, the False Discovery Rate (FDR) is estimated by permutation: (i) the null distribution is simulated by shuffling MS Severity Scores, recalculating Wilcoxon p-values, and repeating the process 10,000 times; (ii) the FDR is computed as follows for every p-value threshold a: FDR=min(1, p.m/R), where R is the number of positives at level a (number of SNPs with a p-value smaller than α), m is the number of tests performed (number of scanned SNPs), and p is the probability to have a p-value smaller than α under the null hypothesis, as estimated by the previous step of permutations^{30,31}.

Genomic Analysis

SNPs were located on the NCBI v36 human genome sequence. Gene structure (exons and introns) annotations were taken from ENSEMBL release 43³². Haplotypes and LD matrices were computed using HaploView³³ using the solid spine of LD method with a 0.8 D′ extension cut-off.

Results

Over the 1,040 patients, the MSSS was on average 4.42 (standard deviation 2.79) spanning from 0.086 to 9.964 (see global distribution in FIG. 1). Out of the 497,641 SNPs, 105,035 (21%) survive the filtering criteria and are used for analysis, covering 63% of the genome.

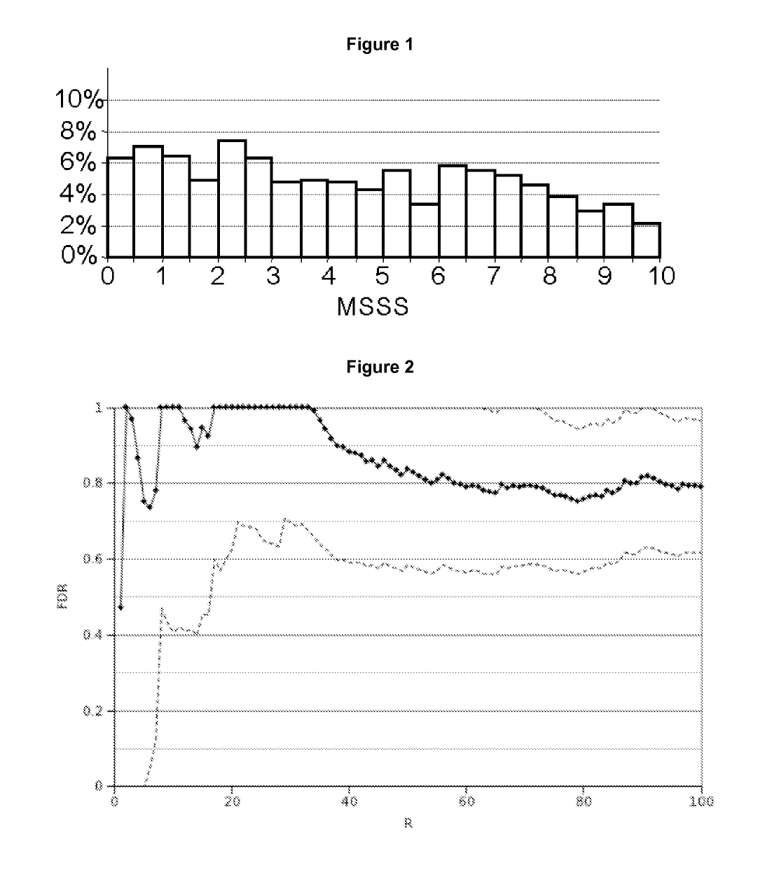

The FDR of observed results was estimated with 10,000 rounds of MSSS shuffling and plotted in FIG. 2 for the 100 smallest p-values. The FDR starts high (around 50%), rises quickly to an 80% plateau and then converges slowly towards 1. When considering the lower boundary of the 95% confidence interval, a 40% FDR threshold selected 14 SNPs (Table 3). These SNPs correspond to frequent genotypes (as ensured by the initial 30% MAF filter) and were all under Hardy-Weinberg Equilibrium. Selection corresponds to a severity p-value cut-off of 1.4e-4. The correlation between genotypes and MSSS is illustrated in FIG. 3.

In classical categorical approaches, the MSSS scale is separated in categories, for instance mild and severe forms of MS, and classical association studies are performed to detect genotype differences between these two categories. When applied to our data set, using for instance two groups of 501 mild MS forms (MSSS<4) and 356 severe MS forms (MSSS>6), we failed to detect any significant associated SNP after multiple-testing correction by FDR³¹. For instance, the SNP rs7221818 (ranked 2 in our continuous approach, see (Table 3) was ranked 67 in the categorical approach (genotypic p-value=6.7e-4) and the FDR for this selection was estimated at 80%. Only the first-ranked SNP rs6552511 is retrieved by categorical approaches, using various MSSS thresholds (data not shown). Once these 14 SNPs have been selected by the continuous scan approach, it is however possible to analyze them in terms of classical categorical relative risks and odds ratios: 9 of the minor genotypes are associated with higher MSSS (relative risks range from 1.5 to 2.3) and 5 are associated with lower MSSS (risks range from 0.4 to 0.8, see tables for details).

These SNPs according to the invention are mapped onto the human genome sequence and compared with gene annotations of ENSEMBL. Mapping details are presented in Table 4. Two SNPs (rs6552511 and rs7221818) are located in desert regions (the closest gene is located more than 100 kb away). The other 12 SNPs fall within or less than 100 kb away from 8 genes. Some of these genes (XYLT1, HIF1AN and MGAT5) are represented by several SNPs that define Linkage Disequilibrium (LD) severity blocks within genes. The three markers located 3′ of HIF1AN on chromosome 10 are in a LD block that does not contain any part of the HIF1AN gene structure (the block is 50 kb away from the HIF1AN stop codon) or any known HIF1AN regulatory region. The rs1078922 SNP is located 22 kb 5′ of the MTPN gene. Other SNPs fall in introns of the assigned genes: first intron of XYLT1 (2 SNPs), second intron of MGAT5 (2 SNPs), eighth intron of MEGF11, third intron of FGF14, seventh intron of PDE9A, and second intron of CDH13.

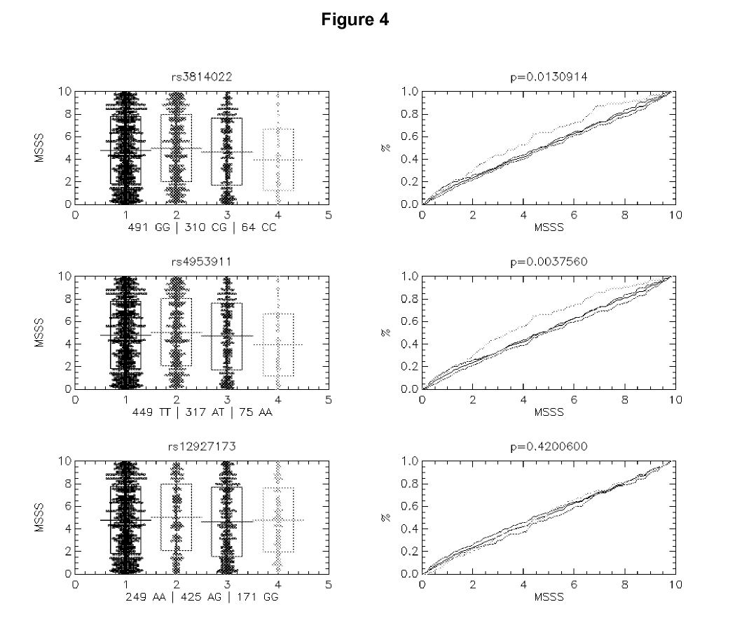

Signals in XYLT1 and MGAT5 were replicated because (i) those signals are represented by multiple SNPs in LD, which can be considered as a technical replication per se and (ii) these two genes encode for glycosylation enzymes and are biologically interesting candidates (see Discussion). Three SNPs were chosen in the two genes: rs12927173 in XYLT1, and rs3814022 and rs4953911 in MGAT5 (a second SNP, rs2059283, was chosen in XYLT1 but the manufacturer was unable to deliver primers). The p-values of these 3 SNPs in the replication dataset (n =873) are respectively 0.42, 1.31e-2 and 3.76e-3 (FIG. 4 and Table 5). The association with MS severity is then replicated in this independent dataset for MGAT5 SNPs. Overall p-values on both datasets are 2.81e-6 and 1.54e-7 for rs3814022 and rs4953911 respectively. For the SNP in XYLT1 (rs12927173), the association is not reproduced in the replication dataset (p=0.42). However the overall p-value on both datasets is still significant (p=1.88e-4).

We have performed a whole-genome scan analysis of over 1,000 MS patients in order to identify markers associated with disease severity. The overall process has led to the identification of 2 markers in un-annotated regions, 3 SNPs in a LD block close to the HIF1AN gene, 1 SNP in the 5′ region of MTPN, and 8 markers inside 6 other genes. Three markers in two genes have been selected and genotyped in an independent replication population, leading to the confirmation of the association of MGAT5 with disease severity. We discuss here the clinical and methodological choices that have made these results possible, and then focus on the biological relevance of selected and replicated severity genes.

There is no consensus method for measuring progression in MS using single, cross-sectional assessments of disability. The MSSS has been recently developed as a powerful method for comparing disease progression in genetic association studies. It adjusts the widely accepted measure of disability, the EDSS, for disease duration comparing an individual's disability with the distribution of scores in cases having equivalent disease duration. The MSSS is potentially superior to the non-linear EDSS for statistical evaluations, as it combines EDSS and disease duration in one variable that is normally distributed. In our three populations, the MSSS distribution it is not homogeneous. This can be explained by different composition of the populations in terms of disease courses, and also by the known inter-observer variability (since the collections come from three different hospitals). This heterogeneity in disability measure assessments might have a significant impact on association results, especially if using arbitrary MSSS cut-off thresholds to define categories.

Previously published severity studies (of candidate genes) classically implement association tests between mild and severe MS sub-populations. In our case, similar categorical approaches using different MSSS thresholds have failed to detect any significantly associated marker. Using cut-off values on EDSS (or derived) scores is probably too arbitrary and inadequate for defining homogeneous severity subgroups, as EDSS only partially (and sometimes subjectively) reflects MS prognosis. With clinical scores, continuous approaches appear then more suitable. For the scan, we have chosen to discard heterozygotes and perform two-sample U-tests between MS patients that are homozygotous for every SNP. It has two theoretical advantages over a classical linear regression approach on 3 samples. First it does not assume that heterozygote patients have an intermediate MS severity (between the severity of the two homozygote groups), which would be the case in an additive model of severity risk. Our approach theoretically allows the detection of dominant or recessive transmission modes for the risk alleles. Second, the Wilcoxon rank-sum test is a non-parametric test: it applies for non-Gaussian MSSS distributions. As a counterpart, this method is probably underpowered for rare markers. We then focused on frequent markers (MAF>30%) for which the frequency of the minor genotype is greater than 9% under Hardy-Weinberg Equilibrium and is well represented in our screened population (n>100). This filtering dramatically reduced the number of analyzed SNPs (down to 105,035) while maintaining reasonable genome coverage (63%). Bigger sample size would be required to investigate less frequent markers with this method (e.g. 2,500 individuals for 20% markers, 10,000 for 10% markers). We can see a posteriori that the MSSS distribution in the whole population is not uniform and that generally the MSSS distributions per SNP genotypes is not Gaussian (FIG. 1 and SNP examples FIG. 3). Finally, it is important to take into account the multiple-testing problem. With conservative family-wise error rate estimation methods (like the Bonferroni correction), there is no SNP selected, meaning we are not able to select a set of markers for which we estimate there is no false-positives. We have preferred to use FDR estimation to control for the multiple-testing because it is more flexible (it allows for a given proportion of false-positives, not necessarily 0%) and it takes into account the dependency between markers³¹.

The FDR-controlled approach has resulted in the selection of 14 markers. The two first-ranked SNPs display important minor genotype frequency differences between mild (MSSS<2) and severe (MSSS>8) clinical outcomes (relative risks are around 2.2) and are then markers of interest for MS severity. They are however located in unannotated genomic region and it is therefore impossible to make hypotheses on their functional impact on disease prognosis. Other SNPs fall inside or close to annotated genes. Among them, MGAT5 is of particular biological interest. The MGAT5 (also known as GNT-V) gene encodes the beta-1,6 N-acetyl-glucosaminyltransferase, an enzyme involved in the synthesis of beta-1,6 GlcNAc-branched N-linked glycans attached to cell surface and secreted glycoproteins. In mice, MGAT5 deficiency has a protective role in tumor growth³⁴ and is associated with enhanced susceptibility to experimental autoimmune encephalomyelitis (EAE) compared to wild-type The The MGAT5 deficiency increases the number of T-cell receptors recruited to the antigen-presenting surface, thereby reducing the requirement for CD28 co-receptor engagement. CD28 and MGAT5 function as opposing regulators of T-cell activation thresholds and susceptibility to immune disease. Association of CD28 with MS severity had previously been tested and shown to be non significant²¹. Moreover, the expression of beta-1,6 GlcNAc-branched N-linked glycans selectively inhibits Th1 cell differentiation and enhances the polarization of Th2 cells³⁶. Deficient glycosylation has been also observed in lymphomonocytes from MS patients: a decrease of GCNT1 (another glucosaminyltransferase) activity by 25-30% is correlated with the occurrence of acute clinical phases of MS and the presence of active lesions in relapsing-remitting We We therefore support here the association of MGAT5 and more generally of the GlcNAc-branched N-linked glycans with MS prognosis. Like MGAT5, XYLT1 (xylosyltransferase I, XT-I) is an enzyme implicated in glyscosylation. XYLT1 is the chain-initiating enzyme involved in the biosynthesis of glycosaminoglycan (GAG)-containing proteoglycans. Proteoglycans, a large group of glycoproteins, are of two main types, chondroitin sulfate (CSPGs) and heparin sulfate (HSPGs). Most CSPGs are secreted from cells and participate in the formation of the extracellular matrix (ECM). CSPGs are the most abundant type of proteoglycans expressed in the mammalian CNS and mainly act as barrier molecules affecting axon growth, cell migration and plasticity, particularly through their GAG-chains. A lesion to the adult CNS provokes the formation of a glial scar, which consists of proliferating and migrating glial cells (mainly reactive astrocytes, microglia and oligodendrocyte precursors) that upregulate several ECM molecules, including CSPGs. The proteoglycans of the glial scar might play a protective role, but the glial scar and its associated CSPGs are one of the main impediments to axon regeneration of injured CNS neurons³⁸. In MS, alteration of ECM molecules have been reported and excessive production and deposition of basement membrane constituents in active MS lesions have been shown and may contribute to axonal loss³⁹. Thus, because XYLT1 initiates GAG-chain elongation and synthesis of CSPGs, two teams have developed a DNA enzyme which target the mRNA of this enzyme and show a reduction of CSPGs^{40,41}. In addition to this link with MS, XYLT1 has shown increased activity in the serum of patients with systemic sclerosis that correlates with clinical classification⁴². Other genes assigned to the selected severity markers are HIF1AN (inhibitor of the Hypoxia-Inducible Factor 1 alpha), MEGF11 (multiple EGF-like domains 11), FGF14 (fibroblast growth factor 14), PDE9A (phosphodiesterase 9A), MTPN (myotrophin) and CDH13 (cadherin 13). No significant marker has been found in the previously reported regions associated with MS severity²³⁻²⁶.

Because MGAT5 and XYLT1 are biologically relevant candidates that share similar glycosylation roles, we decided to replicate the experiment of an independent population. As a result, two SNPs in MGAT5 were clearly confirmed and one SNP in XYLT1 was not found associated in the replication dataset.

In conclusion, the first genome-wide MS severity scan we have performed has led to the hypothesis-free identification of markers associated with disease prognosis. The understanding of molecular mechanisms underlying the disease progression is a crucial point that needs to be addressed in parallel with the search for susceptibility factors. Two of the main identified genes, MGAT5 and XYLT1, are involved in glycosylation processes, thus confirming the importance of glycan regulation in MS. Among those two, MGAT5 was confirmed in an independent replication dataset, whereas XYLT1 replication led to more conflicting results in our study. Glycans play a pivotal role in modulating molecular interactions in the context of multiple physiologic systems, including immune-defense, and glycosylation has been shown to have a critical role in the overall regulation of the immune response43,44. Protein glycosylation is mechanistically important in the pathogenesis of autoimmune diseases: several evidences support the “Remnant Epitopes Generate Autoimmunity (REGA) model” in MS, rheumatoid arthritis (RA) and diabetes⁴⁵. According to this model, the autoimmune process involves cytokines, chemokines and proteinases that cleave glycoproteins into remnant epitopes that are presented to autoreactive T lymphocytes, maintaining the autoimmune reaction. Examples of substrates yielding such remnant epitopes include myelin basic protein, αB-crystallin and interferon-β in MS, and type II collagen in RA⁴⁶. The REGA model has been tested in vivo with the use of animal model and could have interesting therapeutic implications since inhibition of proteinases, such as gelatinase B for MS or RA, results in beneficial effects^{47,48}.

Tables

TABLE 1
Multiple Sclerosis collections

| Dataset | Origin | #individuals | RR (%) | SP (%) | | PP (%) | |
|---|---|---|---|---|---|---|---|
| Screening | Rennes (France) | 384 | 172 (45%) | 135 | (35%) | 77 | (20%) |
| population | Huddinge (Sweden) | 299 | 194 (65%) | 83 | (28%) | 22 | (7%) |
| | San Raffaele (Italy) | 357 | 228 (64%) | 94 | (26%) | 35 | (10%) |
| | Total | 1,040 | 594 (57%) | 312 | (30%) | 134 | (13%) |
| Replication | Rennes (France) | 184 | 110 (60%) | 44 | (24%) | 30 | (16%) |
| population | Huddinge (Sweden) | 689 | 277 (40%) | 348 | (51%) | 61 | (9%) |
| | Total | 873 | 387 (44%) | 392 | (45%) | 91 | (10%) |

| | | | | | | |
|---|---|---|---|---|---|---|
| Overall total | 1,913 | 981 (51%) | 704 | (37%) | 225 | (12%) |

TABLE 2
Demographic and average clinical characteristics of
MS patients in the screening and replication datasets

| | Screening Population | | | |
|---|---|---|---|---|
| | MS | RR | SP | PP |
| | (n = 1040) | (n = 594) | (n = 312) | (n = 134) |
| Female/male | 704/336 | 427/167 | 201/111 | 76/58 |
| Age (years) | 43.2 | 38.9 | 48.4 | 50.2 |
| Disease duration | 12.3 | 9.3 | 18.4 | 11.4 |
| (years) | | | | |
| Age at disease | 30.9 | 29.6 | 30.0 | 38.8 |
| onset (years) | | | | |
| EDSS | 3.6 | 2.0 | 5.4 | 5.3 |

| | Replication Population | | | |
|---|---|---|---|---|
| | MS | RR | SP | PP |
| | (n = 879) | (n = 387) | (n = 392) | (n = 91) |
| Female/male | 631/242 | 298/89 | 248/144 | 52/39 |
| Age (years) | 52.5 | 45.8 | 57.9 | 57.9 |
| Disease duration | 20.4 | 14.9 | 26.1 | 18.9 |
| (years) | | | | |
| Age at disease | 32.1 | 30.9 | 31.8 | 39.0 |
| onset (years) | | | | |
| EDSS | 4.5 | 2.6 | 6.0 | 5.3 |

TABLE 3
SNPs selected at 40% FDR lower-bound threshold.

| | | Homozygote 1 | | | | Heterozygote | | | | Homozygote 2 | | | | severity |
|---|---|---|---|---|---|---|---|---|---|---|---|---|---|---|
| SNP id | rank | | n | m | sd | | n | m | sd | | n | m | sd | p-value |
| rs6552511 | 1 | TT | 435 | 4.03 | 2.69 | CT | 457 | 4.54 | 2.80 | CC | 129 | 5.35 | 2.77 | 5.11E−06 |
| rs7221818 | 2 | TT | 429 | 4.10 | 2.73 | CT | 475 | 4.51 | 2.82 | CC | 128 | 5.29 | 2.73 | 2.78E−05 |
| rs12927173 | 3 | AA | 267 | 4.96 | 2.70 | AG | 524 | 4.37 | 2.81 | GG | 246 | 3.94 | 2.75 | 3.06E−05 |
| rs2059283 | 4 | AA | 267 | 4.94 | 2.69 | AC | 526 | 4.39 | 2.82 | CC | 246 | 3.94 | 2.75 | 3.61E−05 |
| rs1343522 | 5 | AA | 320 | 4.01 | 2.71 | AG | 518 | 4.43 | 2.73 | GG | 198 | 5.08 | 2.93 | 3.91E−05 |
| rs4953911 | 6 | TT | 423 | 4.70 | 2.85 | AT | 452 | 4.42 | 2.72 | AA | 140 | 3.60 | 2.70 | 4.58E−05 |
| rs4573623 | 7 | AA | 298 | 4.06 | 2.75 | AG | 504 | 4.37 | 2.74 | GG | 214 | 5.08 | 2.86 | 5.68E−05 |
| rs333548 | 8 | CC | 481 | 4.25 | 2.78 | CT | 443 | 4.36 | 2.76 | TT | 114 | 5.39 | 2.80 | 1.03E−04 |
| rs10508075 | 9 | GG | 288 | 4.73 | 2.85 | AG | 538 | 4.55 | 2.79 | AA | 206 | 3.74 | 2.63 | 1.05E−04 |
| rs2839580 | 10 | AA | 363 | 4.78 | 2.86 | AC | 513 | 4.39 | 2.74 | CC | 156 | 3.73 | 2.66 | 1.08E−04 |
| rs2495725 | 11 | GG | 304 | 3.99 | 2.69 | AG | 510 | 4.44 | 2.74 | AA | 200 | 5.05 | 2.95 | 1.16E−04 |
| rs3814022 | 12 | GG | 491 | 4.65 | 2.82 | CG | 434 | 4.39 | 2.74 | CC | 107 | 3.53 | 2.68 | 1.20E−04 |
| rs1078922 | 13 | AA | 352 | 4.09 | 2.68 | AG | 470 | 4.40 | 2.82 | GG | 192 | 5.03 | 2.81 | 1.28E−04 |
| rs4315313 | 14 | CC | 426 | 4.70 | 2.83 | CT | 457 | 4.41 | 2.75 | TT | 127 | 3.60 | 2.72 | 1.30E−04 |

n: number of individuals having this genotype; m and sd: MSSS average and standard deviation for these people. The p-value refers to the rank-sum test performed on homozgygote categories (heterozygotes are not used, see text).

TABLE 4
Genomic location of selected severity SNPs.

| | | chromo- | | | |
|---|---|---|---|---|---|
| SNP id | rank | some | position | MAF | Closest gene |
| rs6552511 | 1 | 4q34 | 182,688,603 | 35% | (desert) |
| rs7221818 | 2 | 17p13 | 5,742,055 | 35% | (desert) |
| rs12927173 | 3 | 16p13.1 | 17,378,835 | 49% | XYLT1 (intron) |
| rs2059283 | 4 | 16p13.1 | 17,377,011 | 49% | XYLT1 (intron) |
| rs1343522 | 5 | 10q24 | 102,358,165 | 44% | 58 kb 3′ of |
| | | | | | HIF1AN |
| rs4953911 | 6 | 2q21 | 134,785,280 | 36% | MGAT5 (intron) |
| rs4573623 | 7 | 10q24 | 102,361,387 | 46% | 61 kb 3′ of |
| | | | | | HIF1AN |
| rs333548 | 8 | 15q22 | 64,032,567 | 32% | MEGF11 (intron) |
| rs10508075 | 9 | 13q32 | 101,237,200 | 46% | FGF14 (intron) |
| rs2839580 | 10 | 21q22 | 43,030,176 | 40% | PDE9A (intron) |
| rs2495725 | 11 | 10q24 | 102,354,010 | 45% | 54 kb 3′ of |
| | | | | | HIF1AN |
| rs3814022 | 12 | 2q21 | 134,764,405 | 31% | MGAT5 (intron) |
| rs1078922 | 13 | 7q33 | 135,334,939 | 42% | 22 kb 5′ of |
| | | | | | MTPN |
| rs4315313 | 14 | 16q23 | 81,644,234 | 35% | CDH13 (intron) |

TABLE 5
Replication of severity markers in independent samples

| | | Major homozygote | | | Heterozygote | | | Minor homozygote | | | |
|---|---|---|---|---|---|---|---|---|---|---|---|
| | | # | MSSS | MSSS | # | MSSS | MSSS | # | MSSS | MSSS | |
| SNP | Dataset | samples | mean | sd | samples | mean | sd | samples | mean | sd | p-value |
| rs3814022 | Screen | 491 | 4.65 | 2.82 | 434 | 4.39 | 2.74 | 107 | 3.53 | 2.68 | 1.20E−04 |
| | Replic. | 491 | 4.99 | 2.99 | 310 | 4.66 | 2.99 | 64 | 3.97 | 2.72 | 1.31E−02 |
| | Total | 982 | 4.82 | 2.91 | 744 | 4.50 | 2.84 | 171 | 3.69 | 2.70 | 2.81E−06 |
| rs4953911 | Screen | 423 | 4.70 | 2.85 | 452 | 4.42 | 2.72 | 140 | 3.60 | 2.70 | 4.58E−05 |
| | Replic. | 449 | 5.06 | 2.99 | 317 | 4.69 | 2.99 | 75 | 3.95 | 2.77 | 3.76E−03 |
| | Total | 872 | 4.88 | 2.93 | 769 | 4.53 | 2.84 | 215 | 3.72 | 2.72 | 1.54E−07 |
| rs12927173 | Screen | 267 | 4.96 | 2.70 | 524 | 4.37 | 2.81 | 246 | 3.94 | 2.75 | 3.06E−05 |
| | Replic. | 249 | 5.01 | 2.95 | 425 | 4.66 | 3.07 | 171 | 4.80 | 2.83 | 0.42 |
| | Total | 516 | 4.98 | 2.82 | 949 | 4.50 | 2.93 | 417 | 4.29 | 2.81 | 1.88E−04 |

### REFERENCES

1. Dyment D A, Ebers G C, Sadovnick A D. Genetics of multiple sclerosis. Lancet Neurol. 2004; 3:104-110

2. Hafler D A, Compston A, Sawcer S et al. Risk alleles for multiple sclerosis identified by a genomewide study. N Engl J Med. 2007; 357

3. Lincoln M R, Montpetit A, Cader M Z et al. A predominant role for the HLA class II region in the association of the MHC region with multiple sclerosis. Nat Genet. 2005; 37:1108-1112

4. Compston A, Sawcer S. Genetic analysis of multiple sclerosis. Curr Neurol Neurosci Rep. 2002; 2:259-266

5. Fogdell-Hahn A, Ligers A, Gronning M et al. Multiple sclerosis: a modifying influence of HLA class I genes in an HLA class II associated autoimmune disease. Tissue Antigens. 2000; 55:140-148

6. Harbo H F, Lie B A, Sawcer S et al. Genes in the HLA class I region may contribute to the HLA class Il-associated genetic susceptibility to multiple sclerosis. Tissue Antigens. 2004; 63:237-247

7. Yeo T W, De Jager P L, Gregory S G et al. A second major histocompatibility complex susceptibility locus for multiple sclerosis. Ann Neurol. 2007; 61:228-236

8. Gregory S G, Schmidt S, Seth P et al. Interleukin 7 receptor alpha chain (IL7R) shows allelic and functional association with multiple sclerosis. Nat Genet. 2007;

9. Zhang Z, Duvefelt K, Svensson F et al. Two genes encoding immune-regulatory molecules (LAG3 and IL7R) confer susceptibility to multiple sclerosis. Genes Immun. 2005; 6:145-152

10. Lundmark F, Duvefelt K, Hillert J. Genetic association analysis of the interleukin 7 gene (IL7) in multiple sclerosis. J Neuroimmunol. 2007; 192:171-173

11. Hensiek A E, Seaman S R, Barcellos L F et al. Familial effects on the clinical course of multiple sclerosis. Neurology. 2007; 68:376-383

12. Rasmussen H B, Clausen J. Genetic risk factors in multiple sclerosis and approaches to their identification. J Neurovirol. 2000; 6 Suppl 2:S23-S27

13. Kurtzke J F. Rating neurologic impairment in multiple sclerosis: an expanded disability status scale (EDSS). Neurology. 1983; 33:1444-1452

14. Roxburgh R H, Seaman S R, Masterman T et al. Multiple Sclerosis Severity Score: using disability and disease duration to rate disease severity. Neurology. 2005; 64:1144-1151

15. Kantarci O H, de A M, Weinshenker B G. Identifying disease modifying genes in multiple sclerosis. J Neuroimmunol. 2002; 123:144-159

16. Burwick R M, Ramsay P P, Haines J L et al. APOE epsilon variation in multiple sclerosis susceptibility and disease severity: some answers. Neurology. 2006; 66:1373-1383

17. Santos M, do Carmo C M, Edite R M et al. Genotypes at the APOE and SCA2 loci do not predict the course of multiple sclerosis in patients of Portuguese origin. Mult Scler. 2004; 10:153-157

18. Lindquist S, Schott B H, Ban M et al. The BDNF-Val66Met polymorphism: implications for susceptibility to multiple sclerosis and severity of disease. J Neuroimmunol. 2005; 167:183-185

19. Kroner A, Vogel F, Kolb-Maurer A et al. Impact of the Asp299Gly polymorphism in the toll-like receptor 4 (tlr-4) gene on disease course of multiple sclerosis. J Neuroimmunol. 2005; 165:161-165

20. Hensiek A E, Roxburgh R, Meranian M et al. Osteopontin gene and clinical severity of multiple sclerosis. J Neurol. 2003; 250:943-947

21. van V T, Crusius J B, van W L et al. CTLA-4 and CD28 gene polymorphisms in susceptibility, clinical course and progression of multiple sclerosis. J Neuroimmunol. 2003; 140:188-193

22. Schreiber K, Otura A B, Ryder L P et al. Disease severity in Danish multiple sclerosis patients evaluated by MRI and three genetic markers (HLA-DRB1*1501, CCR5 deletion mutation, apolipoprotein E). Mult Scler. 2002; 8:295-298

23. Mann C L, Davies M B, Stevenson V L et al. Interleukin 1 genotypes in multiple sclerosis and relationship to disease severity. J Neuroimmunol. 2002; 129:197-204

24. Almeras L, Meresse B, Seze J et al. Interleukin-10 promoter polymorphism in multiple sclerosis: association with disease progression. Eur Cytokine Netw. 2002; 13:200-206

25. Goertsches R, Comabella M, Navarro A et al. Genetic association between polymorphisms in the ADAMTS14 gene and multiple sclerosis. J Neuroimmunol. 2005; 164:140-147

26. Mead R J, Neal J W, Griffiths M R et al. Deficiency of the complement regulator CD59a enhances disease severity, demyelination and axonal injury in murine acute experimental allergic encephalomyelitis. Lab Invest. 2004; 84:21-28

27. McDonald W I, Compston A, Edan G et al. Recommended diagnostic criteria for multiple sclerosis: guidelines from the International Panel on the diagnosis of multiple sclerosis. Ann Neurol. 2001; 50:121-127

28. Lublin F D, Reingold S C. Defining the clinical course of multiple sclerosis: results of an international survey. National Multiple Sclerosis Society (USA) Advisory Committee on Clinical Trials of New Agents in Multiple Sclerosis. Neurology. 1996; 46:907-911

29. Wilcoxon F. Individual comparisons by ranking methods. Biometrics. 1945; 1:80-83 30. Storey J D, Tibshirani R. Statistical significance for genomewide studies. Proc Natl Acad Sci U S A. 2003; 100:9440-9445

31. Forner, K., Lamarine, M., Guedj, M., Dauvillier, J., and Wojcik, J. Universal false discovery rate estimation methodology for genome-wide association studies. Human Heredity 2007. Ref Type: In Press

32. Birney E. Ensembl 2007. Nucleic Acids Res. 2007; 35:610-617 33. Barrett J C, Fry B, Mailer J et al. Haploview: analysis and visualization of LD and haplotype maps. Bioinformatics. 2005; 21:263-265

34. Granovsky M, Fata J, Pawling J et al. Suppression of tumor growth and metastasis in Mgat5-deficient mice. Nat Med. 2000; 6:306-312

35. Demetriou M, Granovsky M, Quaggin S et al. Negative regulation of T-cell activation and autoimmunity by Mgat5 N-glycosylation. Nature. 2001; 409:733-739

36. Morgan R, Gao G, Pawling J et al. N-acetylglucosaminyltransferase V (Mgat5)-mediated N-glycosylation negatively regulates Th1 cytokine production by T cells. J Immunol. 2004; 173:7200-7208

37. Orlacchio A, Sarchielli P, Gallai V et al. Activity levels of a beta1,6 N-acetylglucosaminyltransferase in lymphomonocytes from multiple sclerosis patients. J Neurol Sci. 1997; 151:177-183

38. Carulli D, Laabs T, Geller H M et al. Chondroitin sulfate proteoglycans in neural development and regeneration. Curr Opin Neurobiol. 2005; 15:116-120

39. van H J, Bo L, Dijkstra C D et al. Extensive extracellular matrix depositions in active multiple sclerosis lesions. Neurobiol Dis. 2006; 24:484-491

40. Grimpe B, Silver J. A novel DNA enzyme reduces glycosaminoglycan chains in the glial scar and allows microtransplanted dorsal root ganglia axons to regenerate beyond lesions in the spinal cord. J Neurosci. 2004; 24:1393-1397

41. Grimpe B, Pressman Y, Lupa M D et al. The role of proteoglycans in Schwann cell/astrocyte interactions and in regeneration failure at PNS/CNS interfaces. Mol Cell Neurosci. 2005; 28:18-29

42. Gotting C, Sollberg S, Kuhn J et al. Serum xylosyltransferase: a new biochemical marker of the sclerotic process in systemic sclerosis. J Invest Dermatol. 1999; 112:919-924

43. Garcia G G, Berger S B, Sadighi Akha A A et al. Age-associated changes in glycosylation of CD43 and CD45 on mouse CD4 T cells. Eur J Immunol. 2005; 35:622-631

44. Grabie N, Delfs M W, Lim Y C et al. Beta-galactoside alpha2,3-sialyltransferase-I gene expression during Th2 but not Th1 differentiation: implications for core2-glycan formation on cell surface proteins. Eur J Immunol. 2002; 32:2766-2772

45. Descamps F J, Van den Steen P E, Nelissen I et al. Remnant epitopes generate autoimmunity: from rheumatoid arthritis and multiple sclerosis to diabetes. Adv Exp Med Biol. 2003; 535:69-77

46. Opdenakker G, Dillen C, Fiten P et al. Remnant epitopes, autoimmunity and glycosylation. Biochim Biophys Acta. 2006; 1760:610-615

47. Dubois B, Masure S, Hurtenbach U et al. Resistance of young gelatinase B-deficient mice to experimental autoimmune encephalomyelitis and necrotizing tail lesions. J Clin Invest. 1999; 104:1507-1515

48. Itoh T, Matsuda H, Tanioka M et al. The role of matrix metalloproteinase-2 and matrix metalloproteinase-9 in antibody-induced arthritis. J Immunol. 2002; 169:2643-2647

49. Kurzke J. F., Neuroepidemiology, 1991, 10: 1-8

50. Kurzke J. F., Neurology, 1983, 33: 1444-1452

51. McDonald W. I et al., Ann. Neurol., 2001, 50: 121-127

52. Polman C. H. et al., Ann. Neurol. 2005, 58 : 840-846

1-15. (canceled)

16. A method for genotyping comprising the steps of:
a) using a nucleic acid isolated from a sample of an individual; and
b) determining the type of nucleotide in SNP rs3814022, rs4953911, rs2059283, rs12927173, rs2495725, rs1343522, rs4573623, rs333548, rs10508075, rs2839580, rs2495725, rs3814022, rs1078922, and/or rs4315313, in one or both alleles of the diallelic marker, and/or in SNPs in Linkage Disequilibrium (LD) with one or more of these SNPs.

17. The method according to claim 16, wherein the identity of the nucleotides at said diallelic markers is determined for both copies of said diallelic markers present in said individual's genome.

18. The method according to claim 16, wherein said determining is performed by a microsequencing assay.

19. The method according to claim 16, further comprising amplifying a portion of a sequence comprising the diallelic marker prior to said determining step.

20. The method according to claim 19. wherein said amplifying is performed by PCR.

21. The method according to claim 16, further comprising the step of correlating the result of the genotyping steps with the severity of the disease Multiple Sclerosis.

22. The method according to claim 16, wherein the presence of a Gin rs3814022, a T in rs4953911, an A in rs2059283, an A in rs12927173, an A in rs2495725, a G in rs1343522, a G in rs4573623, a Tin rs333548, a G in rs10508075, an A in rs2839580, an A in rs2495725, a G in rs3814022, a G in rs1078922, and/or a C in rs4315313 indicates the severity of the disease Multiple Sclerosis in said individual.

23. The method according to claim 16, wherein the SNPs in Linkage Disequilibrium (LD) with one or more of the SNPs are characterized by a LD correlation coefficient r² greater than 0.8 in at least one population of at least 100 individuals.

24. A composition comprising one or more SNPs selected from the group consisting of rs3814022, rs4953911, rs2059283, rs12927173, rs2495725, rs1343522, rs4573623, rs333548, rs10508075, rs2839580, rs2495725, rs3814022, rs1078922, rs4315313, or SNPs in Linkage Disequilibrium (LD) with one or more of these SNPs.

25. A method which is indicative of the severity of the disease Multiple Sclerosis in an individual comprising:
a) using the nucleic acid from a sample of said individual;
b) identifying the presence of a useful genetic marker in said individual by known methods; and
c) based on the results of step b) making a prediction of the severity of the disease Multiple Sclerosis of said individual.

26. The method according to claim 25, wherein the genetic marker is one or more SNPs selected from the group consisting of rs3814022, rs4953911, rs2059283, rs12927173, rs2495725, rs1343522, rs4573623, rs333548, rs10508075, rs2839580, rs2495725, rs3814022, rs1078922, rs4315313, or SNPs in Linkage Disequilibrium (LD) with one or more of these SNPs.

27. The method according to claim 25 wherein the SNPs in Linkage Disequilibrium (LD) with one or more of the SNPs are characterized by a LD correlation coefficient r² greater than 0.8 in at least one population of at least 100 individuals.

28. A method for treating Multiple Sclerosis in an individual in need thereof, the method comprising the steps:
a) applying a method according to claim 16;
b) treating said individual with an interferon-beta which individual has been identified as exhibiting one or more of the markers and wherein the severity of Multiple Sclerosis in said individual has been determined.

29. The method according to claim 28, wherein the interferon-beta is interferon-beta 1a or 1b.


SEQUENCE LISTING (14 sequences)

SEQ ID NO: 1 - DNA, 33 bp, Homo sapiens
  variation (17)..(17): y is c or t
cattgcaactcatctayacctgtaactcttgtt

SEQ ID NO: 2 - DNA, 33 bp, Homo sapiens
  variation (17)..(17): y is c or t
tagccgttgttgtccayctcctccaatagaatg

SEQ ID NO: 3 - DNA, 33 bp, Homo sapiens
  variation (17)..(17): r is a or g
ggctggctgtcccgccraacaaagagcctggat

SEQ ID NO: 4 - DNA, 33 bp, Homo sapiens
  variation (17)..(17): m is a or c
ttgaccagccttatcamatctgactgtatttcc

SEQ ID NO: 5 - DNA, 33 bp, Homo sapiens
  variation (17)..(17): r is g or a
cccaaagatgccggacrgataccccaagaggtg

SEQ ID NO: 6 - DNA, 33 bp, Homo sapiens
  variation (17)..(17): w is t or a
gtttataaaaactctcwgaaacctcaaagaaca

SEQ ID NO: 7 - DNA, 33 bp, Homo sapiens
  variation (17)..(17): r is g or a
gaatcaggttctgatcragatccacaaatttta

SEQ ID NO: 8 - DNA, 33 bp, Homo sapiens
  variation (17)..(17): y is t or c
gcaattaccggtaagcyatgagagtagtggggg

SEQ ID NO: 9 - DNA, 33 bp, Homo sapiens
  variation (17)..(17): r is g or a
tgttgctgacaattaarccacatagcatttata

SEQ ID NO: 10 - DNA, 33 bp, Homo sapiens
  variation (17)..(17): m is a or c
ttgcatctttgggttamggctctgctgcccttg

SEQ ID NO: 11 - DNA, 33 bp, Homo sapiens
  variation (17)..(17): r is a or g
agtccctaagtgccacraatgaaaagaagactc

SEQ ID NO: 12 - DNA, 33 bp, Homo sapiens
  variation (17)..(17): s is g or c
tttaattccccacaaasagctgagtggctcttg

SEQ ID NO: 13 - DNA, 33 bp, Homo sapiens
  variation (17)..(17): r is g or a
ggaaaacaaattttccrcttctaaggctgttaa

SEQ ID NO: 14 - DNA, 33 bp, Homo sapiens
  variation (17)..(17): y is c or t
tgaatgagataattcaygtgaggctcttagaaa